(19) United States
(12) Patent Application Publication
(10) Pub. No.: US 2007/0082851 A1
(43) Pub. Date: Apr. 12, 2007

(54) Treatment of a condition in a mammal with administration of Compounds and Methods of Use

(75) Inventor: Yoshitaka Ichikawa, San Diego, CA (US)

Correspondence Address: CATALYST LAW GROUP, APC, 9710 SCRANTON ROAD, SUITE S-170, SAN DIEGO, CA, 92121, US

(73) Assignees: Optimer Pharmaceuticals Inc., San Diego, CA (US); The Scripps Research Institute, La Jolla, CA (US)

(21) Appl. No.: 10/580,512

(22) PCT Filed: Nov. 23, 2004
(86) PCT No.: PCT/US04/39680

§ 371 Date: May 23, 2006

Related U.S. Application Data
(60) Provisional application No. 60/524,698, filed on Nov. 24, 2003.

(51) Int. Cl. A61K 31/7052; A61K 31/7008; A61K 31/13; A61K 31/16

(52) U.S. Cl. 514/25; 514/62; 514/419; 514/629; 514/579

(57) ABSTRACT

This invention relates to methods of treating, preventing, and lessening the severity of conditions or diseases selected from the group consisting of osteoarthritis (OA), rheumatoid arthritis, synovitis, subchondral bone edema, and cartilage degradation (“OA and related disorders”) with administration of an aminosugar derivative and pharmaceutically acceptable salts thereof.

### RELATED APPLICATIONS

This application claims the benefit of U.S. provisional application Ser. No. 60/524,698, filed on Nov. 24, 2003, which is hereby incorporated in its entirety by reference.

### FIELD OF THE INVENTION

This invention relates to methods of treating, preventing, and lessening the severity of conditions or diseases selected from the group consisting of osteoarthritis (OA), rheumatoid arthritis, synovitis, subchondral bone edema, and cartilage degradation (hereinafter, “OA and related disorders”) with administration of an aminosugar derivative and pharmaceutically acceptable salts thereof.

### BACKGROUND OF THE INVENTION

Osteoarthritis (OA) is a common joint disorder with significant societal impact (Lawrence et al., (1998) Arthritis Rheum. 41:778; Gabriel et al., (1997) J. Rheumatol. 24:719; March et al., (1997) Baillieres Clin. Rheumatol. 11:817). Classes of medications used for the treatment of OA include acetaminophens, non-steroidal anti-inflammatory drugs (NSAIDS), injectable intra-articular corticosteroids and hyaluronic acid. These drugs primarily provide pain relief, but have not yet been demonstrated to achieve true remission of the disease by slowing or otherwise halting progression of the disease (Altman et al., (1998) Osteoarthritis Cartilage 6 Suppl. A:22; Hochberg et al., (1995) Arthritis Rheum. 38:1535; Hochberg et al., (1995) Arthritis Rheum. 38:1541). Additionally, many of these treatments have negative side effects.

Many forms of arthritis are treated initially with NSAIDS, sometimes together with other analgesics. Where the disease is not adequately controlled with these agents, disease-modifying (remission-inducing) antirheumatic drugs, such as gold salts, D-penicillamine, antimalarial agents and cytotoxic agents, may be utilized. Ultimately, glucocorticoids may be administered, systemically or by the intra-articular route. Yet, none of these drugs is significantly effective in achieving true remission of the disease in most patients.

Bohne described the use of glucosamine (GlcN) to treat OA (Bohne (1969) Med. Welt 30:1668). Since then, GlcN has gained popularity, and now is commonly used to treat OA patients as a supplement, GlcN salts (sulfate and chloride) are thought to have chondroprotective or disease-modifying properties (Altman et al., (1998) Osteoarthritis Cartilage 6 Suppl. A:22; Lozada et al., (1997) Bull. Rheum. Dis. 46:5; Mevorach et al., (1994) Isr. J. Med. Sci. 30:928), and were originally suggested to promote the repair of damaged cartilage. Several studies have demonstrated that cartilage from patients with OA is characterized by accelerated turnover of the cartilage matrix components and by inadequate repair (Inerot et al., (1978) Biochem. J. 169:143; Dieppe et al., (1995) Acta. Orthop. Scand. (Suppl. 266) 66:1). GlcN has been shown to provide anti-inflammatory activity by a number of different mechanisms. For example, GlcN was shown to provide anti-inflammatory activity by inducing upregulation of glycosaminoglycan (GAG) synthesis.

GlcN-induced upregulation of glycosaminoglycan synthesis represents a complex metabolic process, which is potentially mediated through several mechanisms, such as by GlcN directly entering the GAG biosynthetic pathway and circumventing the negative feedback control from uridine-diphosphate N-acetyl-α-D-glucosamine (Kornfeld et al., (1964) Proc. Natl. Acad. Sci. USA 52:371) and upregulation of TGFα1 production (Kolm-Litty et al., (1998) J. Clin. Invest. 101:160). Recently, a novel mechanism of GlcN-mediated chondroprotection was described, which involves the inhibition of aggrecanase activity in bovine cartilage explants and rat chondrosarcoma cells (Sandy et al., (1998) Biochem. J. 335(Pt 1):59) via suppression of glycosylphosphatidylinositol-linked proteins (Sandy et al., (1999) Arch. Biochem. Biophys. 367:258).

GlcN may inhibit phosphorylation events in the interleukin-1β(IL-1β) signaling cascade, providing anti-inflammatory activity. One of the end-products of the hexosamine pathway, UDP-N-acetyl-α-D-glucosamine, was shown to participate in the dynamic process of protein O-glycosylation, which utilizes serine or threonine residues as anchoring sites (Haltiwanger et al., (1997) Biochem. Biophys. Res. Commun. 231:237). Potentially, O-glycosylation of serine/threonine residues can compete with phosphorylation of the same residues, resulting in impairment of intracellular signal transduction cascades (Chou et al., (1995) Proc. Natl. Acad. Sci. USA 92:4417).

IL-1β is known to induce nitric oxide (NO) production in cultured human articular chondrocytes (Geng et al., (1995) J. Cell. Physiol. 163:545). IL-1β-mediated induction of certain mediators of inflammation, including NO, cyclo-oxygenase-2 enzyme (COX-2) and interleukin-6 (IL-6), is associated with translocation of transcription nuclear factor-κB (NF-κB) dimers from the cytoplasm to the nucleus, where they bind to target genes and regulate their transcription (Chu et al., (1998) Biochem. Biophys. Res. Commun. 248:871; Newton et al., (1997) FEBS Lett. 418:135; Parikh et al., (1997) J: Sur. Res. 69:139). The process of NF-κB activation depends on phosphorylation of two serines (Ser-32 and Ser-36) in the κBα inhibitory protein (IκBα) in the N-terminal regulatory domain of the κBβ inhibitory protein (IκBβ) (Karin (1999) J. Biol. Chem. 274:27339).

Anti-inflammatory mechanisms, besides GlcN-induced upregulation of glycosaminoglycan synthesis, may contribute to GlcN's anti-arthritic activities as well. GlcN showed anti-inflammatory activity and protected rats from paw edema induced by bradykinin, serotonin and histamine (Setnikar et al., (1991) Arzneim-Forsch./Drug Res. 41:157). GlcN also protected animals against serositis induced by carragenan, rat peritonitis induced by formalin, and mouse peritonitis induced by acetic acid (Setnikar et al., (1991) Arzneim-Forsch./DrugRes. 41 :157). GlcN did not suppress cyclooxygenase or proteolytic enzymes in the inflamed rat paw, but it did suppress superoxide generation and lysosomal enzyme activities in rat liver (Setnikar et al., (1991) Arzneim-Forsch./Drug Res. 41:157).

Orally administered GlcN also expressed anti-inflammatory activity in kaolin- or adjuvant-induced arthritis in rats (Setnikar et al., (1991) Arzneim-Forsch./Drug Res. 41:542). However, anti-exudative and anti-inflammatory activities of GlcN were lower when administered orally, as compared to corresponding activities of orally administered acetylsalicylic acid or indomethacin.

Despite these advances towards understanding the signaling pathway(s) involved in OA, and related disorders, the pathway(s) is/are not thoroughly revealed. Thus, there is a need in the prior art to better understand these mechanism(s), and, in turn, more precisely treat OA and related disorders with particular and specific aminosugars.

A number of patents relate to the use of GlcN and N-acetylglucosamine (GlcNAc) for the treatment of specific arthritic conditions. U.S. Pat. No. 3,683,076 (Rovati) discloses the use of GlcN salts for the treatment of OA and rheumatoid arthritis; U.S. Pat. No. 4,870,061 (Speck) discloses the use of GlcNAc for treating degenerative joint diseases via buccal administration; U.S. Pat. No. 5,840,715 and U.S. Pat. No. 6,136,795 (both Florio) disclose the use of GlcNAc sulfate (as one of the components) as a nutritional supplement in a dietary regime to provide relief from arthritis. However, there is a continuing need in the art to discover additional compounds useful for the treatment of OA, and related disorders.

The majority of current medications used for the treatment of OA include acetaminophens, NSAIDS, injectable intra-articular corticosteroids and hyaluronic acid. These drugs are unable to cause true remission of OA and have many negative side effects, which are discussed below. Therefore there is a need for a safer and more effective treatment for OA.

Acetaminophens are analgesics used to treat the pain caused by OA. Acetaminophens do not alter the underlying process of cartilage breakdown that occurs in OA and do not affect inflammation. A possible side effect from the use of acetaminophens includes liver damage, caused by long-term use in high doses (greater than 4,000 mg per day) or at lower doses in people with chronic alcohol use or chronic liver disease.

NSAIDs, such as ibuprofen and naproxen, are used to reduce pain, inflammation, and stiffness caused by OA. Side effects of NSAIDs can include asthma attacks, nausea, stomach pain, stomach bleeding, ulcers, and other problems. Long term use of these drugs may injure the kidney, leading to secondary hypertension.

COX-2 inhibitors (such as Vioxx, Bextra, and Celebrex) are also NSAIDs, and are used to reduce pain, inflammation, and stiffness caused by OA. Although they may not cause stomach ulcers like other NSAIDs, there have been some recent findings that they may have some very serious side effects. Vioxx was recently withdrawn from the market by manufacturer Merck & Co. after research showed that long-term use doubled the risk of heart attack and stroke. Additionally, Pfizer recently disclosed that it will probably add a “black box” warning—the strongest kind—to Bextra's label. According to Pfizer, Bextra can cause a rare but sometimes life-threatening drug reaction called Stevens-Johnson Syndrome in which the skin, mouth, and eyes can become horribly blistered. Other drugs, including Celebrex, can also cause this condition—but it seems to be more common with Bextra than other medications. Patients taking Bextra who develop the condition tend to get it in the first two weeks of treatment. Bextra has also been linked to heart risk in high-risk patients.

Although corticosteroids can be helpful to reduce inflammation, they have some unpleasant side effects, such as secondary hypertension (rapid onset high blood pressure that is usually treatable and is from a recognizable cause), indigestion, increased appetite, weight gain, and nervousness or restlessness. Prolonged use of corticosteroids can lead to Cushing's Syndrome. Cushing's syndrome is the result of excessive amounts of cortisol that can come from medication, such as prednisone. Excess cortisol can cause numerous changes, including: increased fat deposits on the face (moon face), neck and trunk; emotional instability; weight gain; high blood pressure; weakness; diabetes; and osteoporosis. Furthermore, corticosteroid use, such as prednisone, cannot be stopped abruptly because the adrenal gland, which makes natural corticosteroids for the body, is suppressed by long term prednisone administration. If a patient has taken large doses of corticosteroids for a long time, the patient's body may need a year to adjust to gradually decreasing doses.

Due to the many side effects of these current OA treatments, as well as their inability to result in true remission of OA due to their being unable to halt or slow the progression of OA, a safer and more effective treatment is greatly needed.

### SUMMARY OF THE INVENTION

The current invention is directed towards the discovery of structures useful for the treatment of OA and related disorders. Said structures are improvements over the current art wherein treatment compounds include the aminosugar glucosamine (GlcN) and N-acetyl-D-glucosamine (GlcNAc). Said structures have specific moieties lending to their improved properties for the treatment of OA and related disorders. The structures of the current invention include, but are not limited to, derivatives of glucosamine, derivatives of galactosamine, derivatives of cyclitol, and derivatives of iminocyclitol.

One embodiment of the present invention relates to the anti-inflammatory and chondroprotective properties of the structures described above in the Field of the Invention section, and further, of the subset of structures in Table 1, and still further, of the sub-subset of structures in Table 2. According to this embodiment, these aminosugars and glycoproteins may exhibit their anti-inflammatory and chondroprotective properties by interfering with cytokine-inducible gene expression in chondrocytes. Said structures have improved protein (including intra- and extra-cellular receptor) binding, improved penetration of the chondrocytes, when compared to the compounds of the prior art, and increased hydrophobicity. Thus, the structures of the current invention are useful as novel treatments for OA, and related disorders.

A preferred embodiment of the present invention relates to methods of treating, preventing, and lessening the severity of synovitis, subchondral bone edema, and cartilage degradation by administering to a patient a therapeutically effective amount of a compound selected from the group consisting of a compound of the structure of the present invention and a pharmaceutically acceptable salt thereof, such as those found in Table 1 and Table 2, and pharmaceutically acceptable salts thereof. Preferably, a therapeutically effective amount of the structure is intra-articularly administered to a patient. More preferably, a therapeutically effective amount of structure is intra-articularly administered while contained in a matrix as a controlled release formulation.

In another preferred embodiment of the present invention, intra-articularly administering the structures to a patient surprisingly showed unexpected and significant retardation of cartilage degeneration in patients with less severe cartilage degradation and reduction of synovial membrane inflammation on both macroscopic and microscopic levels. The retardation of both cartilage degeneration and reduction of synovial membrane inflammation seen after administration of formulations of the invention makes them therapeutically useful for treating, among other conditions, synovitis, subchondral bone edema, and cartilage degeneration in a patient in need of such treatment.

In another preferred embodiment of the present invention, the invention relates to a method including administering to a patient a composition containing a therapeutically effective amount of the structure, either alone or in combination with an existing anti-inflammatory drug or a hexosaminidase inhibitor. Preferably, methods of administering formulations of the present invention include, but are not limited to, intra-articular, topical, and intra-muscular methods. More preferably, controlled release formulations of structures are intra-articularly administered to patients in need of such treatment.

### DETAILED DESCRIPTION OF THE INVENTION

Abbreviations and Terms

In accordance with the present invention and as used herein, the following terms and abbreviations are defined with the following meanings, unless explicitly stated otherwise. These explanations are intended to be exemplary only. They are not intended to limit the terms as they are described or referred to throughout the specification. Rather, these explanations are meant to include any additional aspects and/or examples of the terms as described and claimed herein.

The following abbreviations are used herein:

BrdU=5-bromo-2′-deoxyuridine
GAGs=glycosaminoglycans;
GalNAc=N-Acetylgalactosamine;
GlcN=glucosamine;
GlcNAc=N-Acetylglucosamine;
HA=hyaluronic acid;
IL-1β=interleukin-1β;
IL-6=interleukin-6;
MMP=matrix metalloproteinase;
NSAID=nonsteroidal anti-inflamnmatory drug;
OA=osteoarthritis;
PBS=phosphate-buffered saline;
PEG=polyethylene glycol;
PMSF=phenylmethylsulfonyl fluoride;
RA=rheumatoid arthritis; and
SGAG=sulfated glycosaminoglycan

The term “active ingredient” refers to a therapeutically effective amount of drug or formulation thereof. Preferably, active ingredients of the present invention are those described in Table 1, structures A-J, and derivatives thereof.

The term “alginate gel” refers to natural polysaccharide polymers comprising 1,4-linked β-D-mannuronic and α-L-guluronic acid residues in varying proportions. Alginate is capable of forming stable gels, particularly in the presence of certain divalent cations, such as calcium, barium, and strontium.

The term “amino acid derivative” refers to an amino acid which has been modified to include different substituents.

The term “aminosugar” refers to any synthetic or naturally occurring sugar wherein one or more carbon atoms are substituted with an amino group (—NR¹R²). Such substitution may occur without regard to orientation or configuration of any asymmetric carbons present in the sugar. Unless stated otherwise, the term “aminosugar” refers to either anomer (α or β) of a cyclic or open chain amino sugar. Aminosugars may be N-substituted with alkyl or acyl group, where one hydrogen atom of a pendant amino group is replaced by an alkyl or acyl moiety (—COR where R=lower alkyl).

The term “aminosugar derivative” refers to a derivative of sugar which has at least one amino substituent.

The term “arthritis” refers to any particular disease characterized by joint inflammation, although the etiology of the inflammation may differ in various conditions. Relatively common arthritic diseases include rheumatoid arthritis, juvenile arthritis, ankylosing spondylitis, psoriatic arthritis and osteoarthritis. These are also referred to as “degenerative joint diseases.”

The terms “articular cartilage” or “cartilage” refer to a substance that covers ends of bones and forms the joint surfaces. Cartilage can withstand compressive forces and creates a low friction surface for a joint to glide on. Articular cartilage comprises chondrocytes and a substrate comprising proteins and glycosaminoglycan polysaccharides.

The term “cartilage degradation” refers to degradation in the tissues comprising cartilage.

The term “chitin” refers to (poly)GlcNAc linked in a β-1,4 fashion. Chitin is found throughout nature, for example in the exoskeletons of insects and crustacea.

The term “chitosan” refers to deacetylated chitin or (poly)N-glucosamine linked in a β-1,4 fashion.

The term “chondrocyte” refers to cells found within articular cartilage. Chondrocytes produce collagen, a gelatinous protein, and proteoglycans, which are glucosaminoglycans linked to proteins (also called mucopolysaccharides).

The term “conjugate” refers to the combination of two or more distinct molecules that are chemically bonded. Bonds characteristic of conjugates according to the invention include, but are not limited to, amides, acetals, thioacetals, esters, and thioesters, or any such bond chiefly formed by treating a reactive carbonyl component with a nucleophile.

The term “continuous release” is used solely to describe a release profile that appears to be monophasic, having a smooth-curved time profile of release. Those of skill in the art will appreciate that the release profile may actually correspond to an exponential or logarithmic time-release profile.

The term “cyclitol” refers to a cycloalkane containing one hydroxyl group on each of three or more ring atoms.

The term “derivative of cyclitol” refers to cyclitols which have been modified to include different substituents, including, but not limited to amino groups.

The term “iminocyclitol” refers to a sugar derivative in which the ring oxygen is substituted with a nitrogen atom.

The term “derivative of iminocyclitol” refer to aminosugars which have been modified and these include the hexosaminidase inhibitor iminocyclitol.

The term “derivative of galactosamine” refers to galactosamine which has been modified to include different substituents, including, but not limited to N-acetylgalactosamine.

The term “derivative of glucosamine” refers to glucosamine which has been modified to include different substituents, including, but not limited to N-acetylglucosamine.

The term “encapsulation efficiency” refers to the amount of a compound or active ingredient encompassed, incorporated, loaded, associated, bound or otherwise entrapped within injectable polymeric gels, liposomes, microspheres, nanoparticles, or the like. In general, “yield” is expressed as a percent encapsulation of the active ingredient.

The term “entrapped” or “encapsulated” refers to any method of formulating an active ingredient, which confines, sequesters, or otherwise inhibits the free dissolution of the active ingredient in a matrix, such as a solution or solid phase. Preferred examples of entrapping or encapsulating active ingredients include, but are not limited to, formulations entrapped in a matrix wherein said matrix is selected from a particle, an implant, or a gel.

The term “glycosaminoglycan” refers to long heteropolysaccharide molecules containing repeating disaccharide units. The disaccharide units may comprise modified amino sugars: D-N-acetylgalactosamine or D-GlcNAc and an uronic acid such as D-glucuronate or L-iduronate. Among other functions, GAGs serve as a lubricating fluid in the joints. Specific GAGs of physiological significance are hyaluronic acid, dermatan sulfate, chondroitin sulfate, heparin, heparan sulfate, and keratan sulfate.

The term “hexosamine” refers to any aminosugar of a six-carbon polyhydroxyalcohol containing either an aldehyde or a ketone group. The term hexosamine comprises aldoses, deoxyaldoses and ketoses, without regard for orientation or configuration of the bonds of the asymmetric carbons. Preferred aminosugars are 2-, 3-, 5- or 6-deoxyketoses, preferably deoxyamino sugars such as, for example, GlcN, mannosamine and galactosamine. More preferably, amino sugars are N-acylated and are selected from deoxyacylamino sugars, such as, for example, GIcNAc, N-acetylmannosamine and GalNAc.

The term “hexosaminidase” refers to any glycosidase enzyme that partially or completely hydrolyzes chitin or chitosan into their respective monosaccharide structural units, e.g., GlcNAc and GlcN. Exemplary enzymes include exo-type beta-D-glucosoaminidase, beta-N-acetylhexosaminidase, chitosanase, chitinase, lysozyme, etc.

The term “hyaluronan” refers to a polymer of repeating molecules of N-acetylglucosamine and glucuronic acid.

The term “hyaluronic acid” refers to a naturally occurring linear polysaccharide (long-chain biological polymer) formed by repeating disaccharide units consisting of D-glucuronic acid β(1-3) N-acetyl-D-glucosamine linked by β(1-4) glycosidic linkages. Hyaluronic acid is commercially available in several molecular weight ranges spanning from about 50,000 Daltons to about 8×10⁶ Daltons. Hyaluronic acid is also available as a sodium salt and is a dried, highly purified substance. Sodium hyaluronate may be stored with a variety of preservatives known in the art including, but not limited to, alkyl-substituted benzoic acid esters, alcohols, conjugates, blends, and mixtures thereof.

The term “IL-1β” refers to interleukin-1β, an immunomodulator that mediates a wide range of immune and inflammatory responses, including the activation of B- and T-cells.

The term “IL-6” refers to interleukin-6, a multifunctional cytokine that is produced by a large variety of cells. IL-6 functions as a regulator of immune response, acute-phase reactions and hematopoiesis.

The term “injectable formulation” refers to a sterile, injectable composition prepared as a liquid solution or suspension. Solid forms suitable for solution in, or suspension in, liquid vehicles prior to injection may also be prepared. The preparation may also be emulsified or the active ingredient entrapped. An injectable formulation may also comprise a variety of preservatives known in the art, including, but not limited to, alkyl-substituted benzoic acid esters, alcohols, conjugates, blends, and mixtures thereof.

The term “injectable polymer gel” refers to a polymeric matrix carrier used to entrap or encapsulate active ingredients of the invention. Polymer-based injectable formulations allow drug dosage and timing to be tailored through the choice and formulation of various active ingredient/polymer combinations. The total dose of medication and the kinetics of release are variables that can be adjusted. For example, by varying the solvent content, copolymer ratio and copolymer molecular weight, and polymer solvent polarity drug delivery parameters can be optimized. Polymer-based systems may also increase the life span of active ingredients. The use of polymeric systems comprising poly lactide and lactide-glycolide copolymers in formulations offers certain advantages such as biocompatability and biodegradability. Injectable polymer gels may be prepared, e.g., processed, mixed, filtered, heated, or sterilized according to processes known in the art.

The term “intra-articular” refers to a method of delivering a drug directly to a joint. Traditional routes of drug delivery, such as for example, oral, intravenous or intramuscular administration, depend upon vascular perfusion of the synovium to carry the drug to the joint. This is inefficient because transynovial transfer of small molecules from the synovial capillaries to the joint space generally occurs by passive diffusion, which becomes less efficient with increasing size of the target molecule. Thus, the access of directing molecules, for example, glucosamine (GlcN), to the joint space is substantially restricted. Intra-articular injection or perfusion of drugs circumvents those limitations.

The term “less severe” refers to a particular grade in cartilage degradation of patient Preferably, less severe grade is in the range of Grade 1 to Grade 3. More preferably, less severe grade is in the range of Grade 1 to Grade 2.

The term “liposome” refers to vesicles that spontaneously form when, for example, phospholipids are dispersed in water or an aqueous medium, and result from the hydrophilic interaction of the lipid head groups with water and the creation of uni- and multilamellar systems (vesicles) resembling biological membranes. In a unilamellar liposome, a bilayer structure forms a hollow spherical shape with the polar sides facing an internal water compartment and external bulk water. Several acceptable methods of forming liposomes are known in the art. In general, multilamellar concentric bilayer vesicles are formed with aqueous layers separating lipid bilayers. This onion-like structure is referred to as multilamellar vesicle (MLV). Smaller unilamellar vesicles (SUVs) may be produced by sonication or extrusion of MLVs under appropriate conditions. Liposomes may be formulated with Chol for added stability and may include other materials, such as neutral lipids, and surface modifiers, such as positively or negatively charged compounds. Preferred liposomes are small unilamellar-bilayered spherical shells. Liposomes can encapsulate both lipophillic and hydrophillic drugs. When prepared by appropriate methods, they can release a drug for an extended duration. In addition, there is no toxicity associated with phospholipids. A variety of natural and synthetic phospholipids are commercially available for the preparation of liposomes. Examples known in the art and described by acronyms include, but are not limited to, DSPC, DSPE, DSPE-Con, DSPG and DPPS.

The term “matrix” refers to a solid, gel or liquid composition capable of entrapping an aminosugar(s), and optional additional materials, such as an anti-inflammatory drug, therein.

The term “microsphere” refers to a polymeric matrix carrier used to entrap or encapsulate active ingredients of the invention. Microsphere-based formulations allow drug dosage and timing to be tailored through the choice and formulation of various active ingredient/polymer combinations. The total dose of medication and the kinetics of release are variables that can be adjusted. For example, by varying the copolymer ratio and copolymer molecular weight, drug delivery parameters can be optimized. Microsphere-based systems may also increase the life span of active ingredients. The use of microspheres comprising lactide-glycolide copolymers in formulations offers certain advantages such as biocompatability and biodegradability. Microspheres may be prepared, e.g., processed, machined, milled, ground, or extruded according to processes known in the art.

The term “osteoarthritis related disorders” refers to the following conditions or diseases: osteoarthritis, rheumatoid arthritis, synovitis, subchondral bone edema, and cartilage degradation.

The terms “pharmaceutically acceptable” or “pharmacologically acceptable” refer to formulations that do not produce an adverse, allergic or other untoward reaction when administered to a mammal as appropriate, (e.g., by a physician or veterinarian).

The terms “polyethylene glycol” and “PEG” refer to a water-soluble polymer comprising submits HO—(CH₂CH₂O)ₙH. PEG may be end-capped with alkyl groups.

The term “polymeric” or “polymeric carrier” refers to hyaluronic acid, polyethylene glycol, copolymers of polyethylene glycol and poly (lactic/glycolic acid), polymers of lactic acid, and copolymers of poly (ethylene glycol-γ-(DL-lactic acid-co-glycolic acid), alginate gels, chitosans, or pharmaceutically acceptable salts thereof.

The term “sustained release” refers to the time period during which a drug is released for availability, or otherwise becomes available for physiological uptake. Periods of sustained release may be preceded by an induction period, during which little or no drug is released, or may be biphasic, comprising an initial time period during which some drug is released, and a second time period during which additional drug is released.

The term “synovitis” means inflammation of the joint lining (synovium). Synovitis is present in a variety ofjoint related conditions, including, but not limited to osteoarthritis, physical or traumatic injury, rheumatoid arthritis and other autoimmune disorders.

The term “therapeutically effective amount” refers to the amount of a biologically active substance necessary to induce a desired pharmacological effect. The amount can vary greatly according to the effectiveness of a particular active substance; the age, weight, and response of the individual; as well as the nature and severity of the individual's symptoms. Accordingly, there is no upper or lower critical limitation with respect to the amount of the active substance. A therapeutically effective amount to be employed in the present invention can readily be determined by those skilled in the art.

This invention relates to methods of treating, preventing, and lessening the severity of conditions or diseases selected from the group consisting of osteoarthritis (OA), rheumatoid arthritis, synovitis, subchondral bone edema, and cartilage degradation (hereinafter, “OA and related disorders”) with administration of an aminosugar and pharmaceutically acceptable salts thereof.

The current invention is directed towards the discovery of structures useful for the treatment of OA and related disorders. Said structures are improvements over the current art wherein treatment compounds include the aminosugar glucosamine (GlcN) and N-acetyl-D-glucosamine (GlcNAc). Said structures have specific moieties lending to their improved properties for the treatment of OA and related disorders. The structures of the current invention include, but are not limited to, derivatives of glucosamine, derivatives of galactosamine, derivatives of cyclitol, and derivatives of iminocyclitol. The related family of aminosugar structures (hereinafter “structures”) shown below are of particular interest. 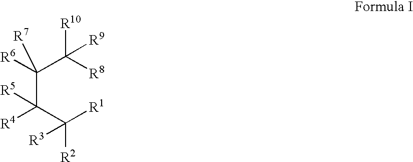
Compounds Claimed Herein Include those of Formula I wherein:
R¹ is: CHO, CH₂OH, or CO₂H;
R² is: H, OH, OR¹¹ (where R¹¹ is ether-linked cyclic or a cyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group, NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R³ is: H, C-linked cyclic or acyclic alkyl, aryl, heterocyclic group, or R², R³═O;
R⁴ is: H, OH, OR¹¹ (where R¹¹ is ether-linked cyclic or a cyclic alkyl, aryl, heterocyclic group), OCOR¹² (where R¹² is cyclic or acyclic allyl, aryl, heterocyclic, or amino acid derivative), Cl, Br, F, SH, SR¹³ (where R¹³ is ether-linked cyclic or acyclic alkyl, aryl, heterocyclic group, NH₂, NR¹⁴R¹⁵ (where R¹⁴ or R¹⁵ is H or ether-linked cyclic or acyclic aLkyl, aryl, heterocyclic group), or NHCOR¹⁶ (where R¹⁶ is cyclic or acyclic alkyl, aryl, heterocyclic, or amino acid derivative);
R⁵ is: H, C-linked cyclic or acyclic alkyl, aryl, heterocyclic group, or R⁴, R⁵═O;
R⁶ is: H, OH, OR¹¹ (where R¹¹ is ether-linked cyclic or a cyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group, NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R⁷ is: H, C-linked cyclic or acyclic alkyl, aryl, heterocyclic group, or R⁶, R⁷═O;
R⁸ is: H, OH, OR¹¹ (where R¹¹ is ether-linked cyclic or a cyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group, NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic allyl, aryl, heterocyclic, or amino acid derivative);
R⁹ is: H, C-linked cyclic or acyclic alkyl, aryl, heterocyclic group, or R⁸, R⁹═O;
R¹⁰ is: H, CH₃, CH₂OH, CH₂OR¹¹ (where R¹¹ is ether-linked cyclic or acyclic alkyl, aryl, heterocyclic group), CH₂OCOR¹² (where R¹² is cyclic or acyclic alkyl, aryl, heterocyclic, or amino acid derivative), CH₂Cl, CH₂Br, CH₂F, CH₂SH, CH₂SR¹³ (where R¹³ is ether-linked cyclic or acyclic alkyl, aryl, heterocyclic group, CH₂NH₂, CH₂NR¹⁴R¹⁵ (where R¹⁴ or R¹⁵ is H or ether-linked cyclic or acyclic alkyl, aryl, heterocyclic group), or CH₂NHCOR¹⁶ (where R¹⁶ is cyclic or acyclic alkyl, aryl, heterocyclic, or amino acid derivative); and
   when a compound with R¹═CHO, it may exist as a cyclic form if R⁸ is OH, SH, NH₂, or NHR¹⁴, or R⁶ is OH, SH, NH₂, or NHR¹⁴. 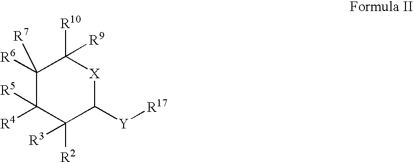
      Compounds Claimed Herein Include those of Formula II wherein:
X is: O, S, CH₂, NH, or NR²⁰ (where R²⁰ is cyclic or acyclic alkyl, aryl, heteroxyclic group);
Y is: O, S, CH₂, or NH;
R¹⁷ is: H, OH, OR¹¹ (where R¹¹ is ether-linked cyclic or a cyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group), NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R² is: H, OH, OR¹¹ (where R¹¹ is ether-linked cyclic or a cyclic alkyl, aryl, heterocyclic group), OCOR¹² (where R¹² is cyclic or acyclic alkyl, aryl, heterocyclic, or amnino acid derivative), Cl, Br, F, SH, SR¹³ (where R¹³ is ether-linked cyclic or acyclic alkyl, aryl, heterocyclic group), NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R³ is: H, C-linked cyclic or acyclic alkyl, aryl, heterocyclic group, or R², R³═O;
R⁴ is: H, OH, OR¹¹ (where R¹¹ is ether-linked cyclic or a cyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group), NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R⁵ is: H, C-linked cyclic or acyclic alkyl, aryl, heterocyclic group, or R⁴, R⁵═O;
R⁶ is: H, OH, OR¹¹ (where R¹¹ is ether-linked cyclic or a cyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group), NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R⁷ is: H, C-linked cyclic or acyclic alkyl, aryl, heterocyclic group, or R⁶, R⁷═O;
R⁹ is: H, C-linked cyclic or acyclic alkyl, aryl, or heterocyclic group; and
R¹⁰ is: H, CH₃, CH₂OH, CH₂OR¹¹ (where R¹¹ is ether-linked cyclic or acyclic alkyl, aryl, heterocyclic group), CH₂OCOR¹² (where R¹² is cyclic or acyclic alkyl, aryl, heterocyclic, or amino acid derivative), CH₂Cl, CH₂Br, CH₂F, CH₂SH, CH₂SR¹³ (where R¹³ is ether-linked cyclic or acyclic alkyl, aryl, heterocyclic group), CH₂NH₂, CH₂NR¹⁴R¹⁵ (where R¹⁴ or R¹⁵ is H or ether-linked cyclic or acyclic alkyl, aryl, heterocyclic group), or CH₂NHCOR¹⁶ (where R¹⁶ is cyclic or acyclic alkyl, aryl, heterocyclic, or amino acid derivative). 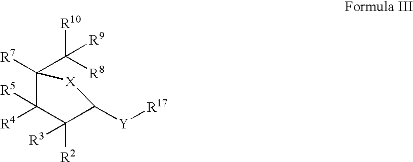
   Compounds Claimed Herein Include those of Formula III wherein:
X is: O, S, CH₂, NH, or NR²⁰ (where R²⁰ is cyclic or acyclic alkyl, aryl, heterocyclic group);
Y is: O, S, CH₂, or NH;
R¹⁷ is: H, OH, OR¹¹ (where R¹¹ is ether-linked cyclic or a cyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group), NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R² is: H, OH, OR¹¹ (where R¹¹ is ether-linked cyclic or a cyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group), NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R³ is: H, C-inked cyclic or acyclic alkyl, aryl, heterocyclic group, or R², R³═O;
R⁴ is: H, OH, OR¹¹ (where R¹¹ is ether-linked cyclic or a cyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group), NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R⁵ is: H, C-inked cyclic or acyclic alkyl, aryl, heterocyclic group, or R⁴, R⁵═O;
R⁷ is: H, C-linked cyclic or acyclic alkyl, aryl, or heterocyclic group;
R⁸ is: H, OH, OR¹¹ (where R¹¹ is ether-linked cyclic or acyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group), NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R⁹ is: H, C-linked cyclic or acyclic alkyl, aryl, or heterocyclic group; and
R¹⁰ is: H, CH₃, CH₂OH, CH₂OR¹¹ (where R¹¹ is ether-linked cyclic or acyclic alkyl, aryl, heterocyclic group), CH₂OCOR¹² (where R¹² is cyclic or acyclic alkyl, aryl, heterocyclic, or amino acid derivative), CH₂Cl, CH₂Br, CH₂F, CH₂SH, CH₂SR¹³ (where R¹³ is ether-linked cyclic or acyclic alkyl, aryl, heterocyclic group), CH₂NH₂, CH₂NR¹⁴R¹⁵ (where R¹⁴ or R¹⁵ is H or ether-linked cyclic or acyclic alkyl, aryl, heterocyclic group), or CH₂NHCOR¹⁶ (where R¹⁶ is cyclic or acyclic alkyl, aryl, heterocyclic, or amino acid derivative). 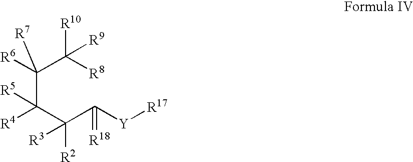
   Compounds Claimed Herein Include those of Formula IV wherein:
Y is: O, S, CH₂, or NH;
R¹⁷ is: H, OH, OR¹¹ (where R¹¹ is ether-linked cyclic or a cyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group), NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R¹⁸ is: H, O, NH, or NR¹⁹ (where R¹⁹ is cyclic or acyclic alkyl, aryl, heterocyclic group or acyl-linked cyclic or acyclic alkyl, aryl, or heterocyclic group);
R² is: H, OH, OR¹¹ (where R¹¹ is ether-linked cyclic or a cyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group, NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R³ is: H, C-linked cyclic or acyclic alkyl, aryl, heterocyclic group, or R², R³═O;
R⁴ is: H, OH, OR¹¹ (where R¹¹ is ether-linked cyclic or a cyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group, NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R⁵ is: H, C-linked cyclic or acyclic alkyl, aryl, heterocyclic group, or R⁴, R⁵═O;
R⁶ is: H, OH, OR¹¹ (where R¹¹ is ether-linked cyclic or a cyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group, NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R⁷ is: H, C-linked cyclic or acyclic alkyl, aryl, heterocyclic group, or R⁶, R⁷═O;
R⁸ is: H, OH, OR¹¹ (where R¹¹ is ether-linked cyclic or a cyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group, NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R⁹ is: H, C-linked cyclic or acyclic alkyl, aryl, heterocyclic group, or R⁸, R⁹═O; and
R¹⁰ is: H, CH₃, CH₂OH, CH₂OR¹¹ (where R¹¹ is ether-linked cyclic or acyclic alkyl, aryl, heterocyclic group), CH₂OCOR¹² (where R¹² is cyclic or acyclic alkyl, aryl, heterocyclic, or amino acid derivative), CH₂Cl, CH₂Br, CH₂F, CH₂SH, CH₂SR¹³ (where R¹³ is ether-linked cyclic or acyclic alkyl, aryl, heterocyclic group, CH₂NH₂, CH₂NR¹⁴R¹⁵ (where R¹⁴ or R¹⁵ is H or ether-linked cyclic or acyclic alkyl, aryl, heterocyclic group), or CH₂NHCOR¹⁶ (where R¹⁶ is cyclic or acyclic alkyl, aryl, heterocyclic, or amino acid derivative).

Compounds with Y—R¹⁷ may exist as a monomeric, oligomeric, or polymeric form linked through the R¹⁷ functionality. These may be conjugated covalently, through R¹⁷ moiety as a linker, to another molecule, including peptide, protein, or particle. These may also be incorporated non-covalently, through R¹⁷ moiety as an anchor, as a form of liposome or particle.

One embodiment of the present invention relates to the anti-inflammatory and chondroprotective properties of the structures described above, and further, of the subset of structures in Table 1, and still further, of the sub-subset of structures in Table 2. According to this embodiment, these aminosugars and glycoproteins may exhibit their anti-inflammatory and chondroprotective properties by interfering with cytokine-inducible gene expression in chondrocytes. Said structures have improved protein (including intra- and extra-cellular receptor) binding, improved penetration of the chondrocytes, when compared to the compounds of the prior art, and increased hydrophobicity. Thus, the structures of the current invention are useful as novel treatments for OA, and related disorders.

A preferred embodiment of the present invention relates to methods of treating, preventing, and lessening the severity of synovitis, subchondral bone edema, and cartilage degradation by administering to a patient a therapeutically effective amount of a compound selected from the group consisting of a compound of the structure of the present invention and a pharmaceutically acceptable salt thereof, such as those found in Table 1 and Table 2, and pharmaceutically acceptable salts thereof. Preferably, a therapeutically effective amount of the structure is intra-articularly administered to a patient. More preferably, a therapeutically effective amount of structure is intra-articularly administered while contained in a matrix as a controlled release formulation.

In another preferred embodiment of the present invention, intra-articularly administering the structures to a patient surprisingly showed unexpected and significant retardation of cartilage degeneration in patients with less severe cartilage degradation and reduction of synovial membrane inflammation on both macroscopic and microscopic levels. The retardation of both cartilage degeneration and reduction of synovial membrane inflammation seen after administration of formulations of the invention makes them therapeutically useful for treating, among other conditions, synovitis, subchondral bone edema, and cartilage degeneration in a patient in need of such treatment.

In another preferred embodiment of the present invention, the invention relates to a method including administering to a patient a composition containing a therapeutically effective amount of the structure, either alone or in combination with an existing anti-inflammatory drug or a hexosaminidase inhibitor. Preferably, methods of administering formulations of the present invention include, but are not limited to, intra-articular, topical, and intra-muscular methods. More preferably, controlled release formulations of structures are intra-articularly administered to patients in need of such treatment.

Formulations and Methods

The present invention relates to the use of novel aminosugars for their anti-inflammatory activity. Accordingly, the following discussion details experiments designed to determine the origin of chondroprotective and anti-inflammatory properties of these aminosugars. From said studies are derived novel structures for the treatment of OA and related disorders. Without limiting the scope of Applicants' discovery, the related family of structures shown below are particularly useful as compounds of the current invention.

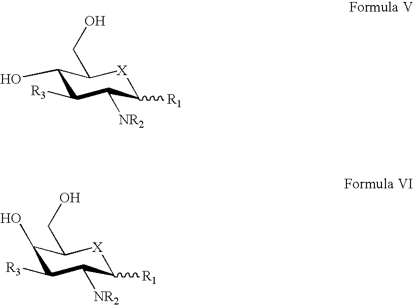

Where X may be O, NH, or CH₂

These (subset of structures A-J) of Formula V and VI are defined by their particular pattern of substituents as shown in Table 1, and referred to above.

TABLE 1
Sub-set of structures (A-J)

| | R¹ | R² | R³ |
|---|---|---|---|
| A | OMe | 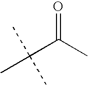 | OH |
| | methoxy | acetyl | hydroxy |
| B | 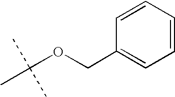 | 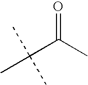 | OH |
| | benzyloxy | acetyl | hydroxy |
| C | 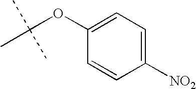 | 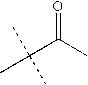 | OH |
| | p-nitrophenoxy | acetyl | hydroxy |
| D | 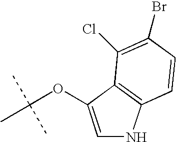 | 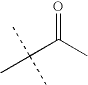 | OH |
| | 5-bromo-4-chloro-3-indolyl | acetyl | hydroxy |
| E | OH | 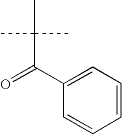 | OH |
| | hydroxy | benzoyl | hydroxy |
| F | OH | 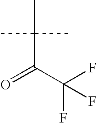 | OH |
| | hydroxy | trifluoroacetyl | hydroxy |
| G | OH | 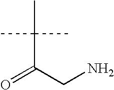 | OH |
| | hydroxy | aminoacetyl | hydroxy |
| H | OH | 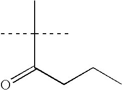 | OH |
| | hydroxy | butyryl | hydroxy |
| I | OH | 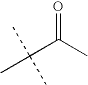 | 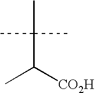 |
| | hydroxy | acetyl | (R)-1-carboxyethyl |
| J | 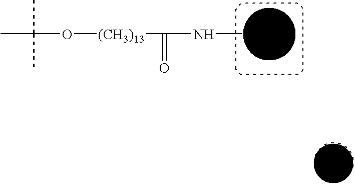 | 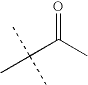 | OH |
| | tetradecanoyl-BSA | acetyl | hydroxy |

Methods for screening compounds and assays measuring methods are published and otherwise well known in the art For example, see PCT publication WO 02/078445 A1, which is incorporated herein by reference in its entirety. The structures of the current invention will be similarly screened, to elucidate the improved properties thereof. The Examples section below lists the various assays that can be performed to screen the structures of the current invention.

Various modifications and alterations of the invention are apparent to those skilled in the art and do not depart from the spirit and scope of the invention. For example, it should be noted that steps recited in any method do not necessarily need to be performed in the order that they are recited. Those of ordinary skill in the art will recognize variations in performing the steps from the order in which they are recited. For further example, in certain embodiments, steps may be performed simultaneously. The foregoing methods should be constructed with these principles in mind.

Studies have uncovered particular structures useful as pharmaceutical compositions for the treatment of OA and related disorders. Without being bound to any theory, said structures and their derivatives are an improvement over the compounds of the prior art in that said structures posses improved hydrophobicity, improved hydrolytic characteristics, improved cell penetration, enhanced protein and receptor (intracellular and extracellular) binding, and improved structure. Furthermore, said structures are useful for elucidating the signaling pathway(s) involved in treating OA and related disorders with aminosugars, which, in turn, allows for a customized selection of specific structures for a more precise treatment of a patient's OA or related disorder.

By way of example only, the following 5 classes of specific structures are of particular interest.

TABLE 2
Sub-subset of structures (Classes 1-5)
Class 1; Simple alkyl glycosides of GlcNAc/GalNAc

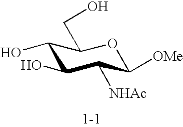

1-1

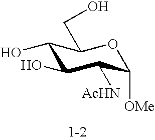

1-2

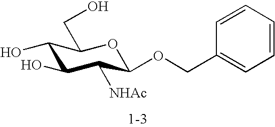

1-3

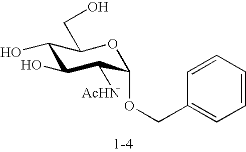

1-4
Class 2: N-Acyl derivatives of Glucosamine (GlcNH₂)

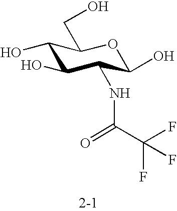

2-1

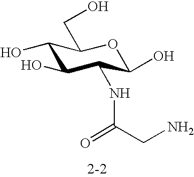

2-2

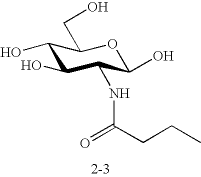

2-3

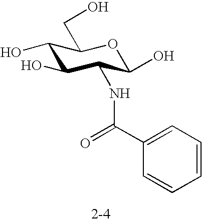

2-4
Class 3; Substrates of hexosaminidase

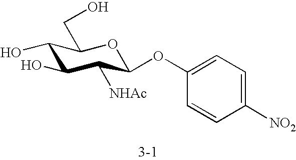

3-1

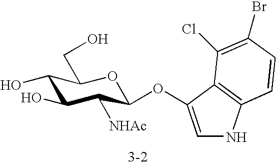

3-2
Class 4: Multi-valent form of GlcNAc

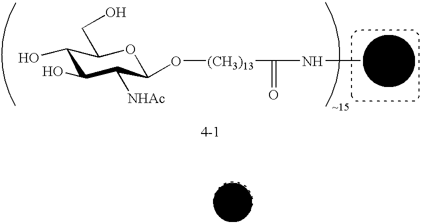

4-1

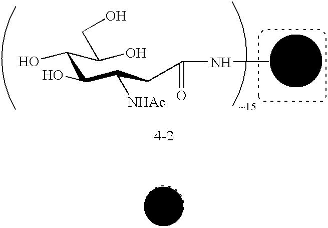

4-2
Class 5; GlcNAc-like molecules (sugar derivatives)

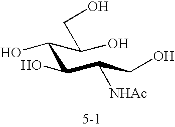

5-1

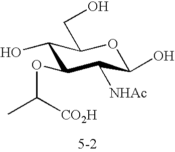

5-2

### Pharmaceutical Formulation and Administration

An aminosugar or derivative thereof, as the active ingredient, can be put in pharmaceutically acceptable formulations, such as those described in Remington's Pharmaceutical Sciences, 18^{th} ed., Mack Publishing Co., Easton, Pa. (1990), incorporated by reference herein, and used for specific treatment of diseases and pathological conditions with little or no effect on healthy tissues. The preparation of a pharmacological composition comprising active ingredients dissolved or dispersed therein need not be limited based on formulation. Such compositions may be prepared as injectable liquid solutions or suspensions. However, solid forms suitable for dissolution, or resuspension, in liquid prior to use can also be prepared. The preparation can also be emulsified.

In a preferred embodiment, the composition is held within a container, which includes a label stating to the effect that the composition is approved by the FDA in the United States (or other equivalent labels in other countries) for treating a disease or condition described herein. Such a container will provide therapeutically effective amount of the active ingredient to be administered to a host.

The particular aminosugars that affect the conditions of interest can be administered to a mammal either alone or in pharmaceutical compositions where it is mixed with suitable carrier(s) or excipient(s). In treating a mammal exlubiting a condition of interest, a therapeutically effective amount of an agent or agents, such as one of the structures of the present invention (including, but not limited to derivatives of glucosamine, derivatives of galactosamine, derivatives of cyclitol, or derivatives of irainocyclitol), is administered. The active ingredient can be mixed with excipients that are pharmaceutically acceptable and compatible with said active ingredient and in amounts suitable for use in the therapeutic methods described herein.

Pharmaceutically acceptable salts can be prepared by standard techniques. For example, the free base form of the compound is first dissolved in a suitable solvent such as an aqueous or aqueous-alcohol solution, containing the appropriate acid. The salt is then isolated by evaporating the solution. In another example, the salt is prepared by reacting the free base and acid in an organic solvent.

Carriers or excipients can be used to facilitate adrninistration of the compound, for example, to increase the solubility of the compound. Examples of carriers and excipients include calcium carbonate, calcium phosphate, various sugars or types of starch, cellulose derivatives, gelatin, vegetable oils, polyethylene glycols, water, saline, dextrose, glycerol, ethanol and physiologically compatible solvents.

Compositions of the present invention can include pharmaceutically acceptable salts of the components therein. Pharmaceutically acceptable salts include acid addition salts (formed with any free amino groups of the aminosugars) that are formed with inorganic acids such as, for example, hydrochloric or phosphoric, sulfuric acids, etc., or such organic acids as acetic, tartaric, mandelic and the like. Salts formed with the free carboxyl groups of the aminosugars can also be derived from inorganic bases such as, for example, sodium, potassium, ammonium, calcium or ferric hydroxides, and such organic bases as isopropylarnine, trimethylamine, 2-aminoethanol, histidine, procaine and the like.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. The data obtained from these cell culture assays and animal studies can be used in formulating a range of dosage for use in human. The dosage of such compounds lies preferably within a range of circulating concentrations that include the ED50 with little or no toxicity. The dosage may vary within this range depending upon the dosage form employed and the route of administration utilized.

For any aminosugar compound used in the methods of the invention, the therapeutically effective dose can be estimated initially from cell culture assays. For example, a dose can be formulated in animal models to achieve a circulating plasma concentration range that includes the IC50 as determined in cell culture (i.e., the concentration of the test compound which achieves a half-maximal disruption of the protein complex, or a half-maximal inhibition of the cellular level and/or activity of a complex component). Such information can be used to more accurately determine useful doses in humans. Levels in plasma may be measured, for example, by HPLC.

Another preferred embodiment of the present invention relates to an improved formulation for the active ingredient, GlcNAc. Encapsulation or entrapment of GlcNAc in liposomes or other entrapping agents modifies its pharmacodynamic profile when intra-articularly injected. Preferably, GlcNAc is entrapped in a matrix. More preferably, GlcNAc in entrapped in a matrix selected from the groups consisting of a particle, an implant, or a gel.

The exact formulation, route of administration and dosage can be chosen by the individual physician in view of the mammal's condition. (See e.g. Fingl et al., in The Pharmacological Basis of Therapeutics, 1975, Ch. 1 p. 1). It should be noted that the attending physician would know how to and when to terminate, interrupt, or adjust administration due to toxicity, or to organ dysfimctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual mammal. A program comparable to that discussed above may be used in veterinary medicine.

Depending on the specific conditions being treated, such agents may be formulated and administered systemically or locally. Techniques for formulation and administration may be found in Remington's Pharmaceutical Sciences, 18th ed., Mack Publishing Co., Easton, Pa. (1990), which is incorporated herein by reference.

For injection, the agents of the invention may be formulated in aqueous solutions, preferably in physiologically compatible buffers such as Hanks's solution, Ringer's solution, or physiological saline buffer.

Use of pharmaceutically acceptable carriers to formulate the compounds herein disclosed for the practice of the invention into dosages suitable for systemic administration is within the scope of the invention. With proper choice of carrier and suitable manufacturing practice, the compositions of the present invention, in particular, those formulated as solutions, may be administered parenterally, such as by intravenous injection.

Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein. In addition to the active ingredients, these pharmaceutical compositions may contain suitable pharmaceutically acceptable carriers comprising excipients and auxiliaries which facilitate processing of the active compounds into preparations which can be used pharmaceutically. The pharmaceutical compositions of the present invention may be manufactured in a manner that is itself known, e.g., by means of conventional mixing, dissolving, granulating, dragee-making, levitating, emulsifyg, encapsulating, entrapping or lyophilizing processes.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

### EXAMPLES

The following examples are provided by way of describing specific embodiments of the present invention without intending to limit the scope of the invention in any way.

### Example 1

Treatment of Cultured Osteoartdritic Human Articular Chondrocytes with Aminosugars

Human osteoarthritic cartilage will be obtained after routine total knee replacements from informed donors. Articular cartilages from the femoral condyles and the tibial plateaus will be aseptically dissected. Cartilage shavings will be harvested and placed in tissue culture medium (DMEM, 10% FBS, Penicillin, Streptomycin) and stored at 4° C. until they are processed.

Chondrocytes will be isolated from the cartilage after sequential digestion with pronase (Roche, 10 g/l) for 30 min and collagenase type IV (Sigma, 1 g/l) for 6 h, both in 0.9% NaCl. Chondrocytes will be grown to confluence in DMEM (BioWhittaker) supplemented with 10% fetal calf serum (BioWhittaker), 60 U/ml penicillin, 60 μg/ml streptomycin and 2 mmol/l glutamine (BioWhittaker) at 37° C. in the presence of 5% CO₂.

Experiments will be performed with first or second passage cells. Chondrocyte proliferation and apoptosis will be determined after incubation of human chondrocytes with different levels of each of the aminosugar derivatives of the present invention, including, but not limited to derivatives of glucosamine, derivatives of galactosamine, derivatives of cyclitol, and derivatives of iminocyclitol. Where indicated, cells will be preincubated with equimolar concentrations of each structure. Cell morphology and viability will be assessed macroscopically by observation with stain and biochemically using BrdU staining.

### Example 2

Effect of Aminosugars on IL-1β Induced .NO in Cultured Chondrocytes

IL-1β induced inflammatory mediator, .NO will be measured according to the methods of Griess reaction (Hevel, et. al. MethlodsEnzymol. 233:250) and via protein ELISA (R&D Systems, Minneapolis, Minn.).

Aminosugars, such as GlcN and GlcNAc, are known to inhibit IL-1β and TNFα induced nitric oxide (.NO) production in osteoarthritic human articular chondrocytes (PCT publication WO 02/078445 A1). Thus, the effect of the aminosugar derivatives of the present invention, including, but not limited to derivatives of glucosamine, derivatives of galactosamine, derivatives of cyclitols, and derivatives of iminocyclitol, on IL-1β induced .NO production will be measured according to the methods of Griess reaction and compared.

In an oxygenated solution .NO decomposes to form NO₂⁻ and NO₃⁻ as shown in equations (1)-(3).
2.NO+O₂→2.NO₂H₂O (1)
.NO+.NO₂→N₂O₃→2NO₂⁻H₂O (2)
2.NO₂→N₂O₄→NO₂⁻+NO₃⁻ (3)

These stable end products can be detected using a discontinuous spectrophotometric assay. Nitrite can be directly detected by observing the magenta-colored azo dye that is formed from NO₂⁻ and the Griess reagent. On the other hand, NO₃⁻ must first be reduced to NO₂⁻ with either nitrate reductase or a copper-plated cadmium column if an in-line low-pressure pumping system is being used. The automated system has been described in detail elsewhere (Green et al. Anal. Biochemn. 126:131), but certain aspects of the system are discussed below.

Cultured cells, such as the osteoarthritic human articular chondrocytes described in Example 1 above, can be plated at 40,000 cells/well in 96-well plates in the presence of 1% FBS. After 48 hours the medium will be changed (this medium should not contain phenol red, as this absorbs in the same region as the product of the Griess reaction). The cells should then be stimulated with 5 ng/ml IL-1β for 24 hours in the presence of the abovementioned aminosugars of the present invention. The .NO production will be detected as NO₂⁻ accumulation in the cell culture supernatants.

Griess reagent should be prepared fresh by mixing equal volumes of 0.1% naphthylethylenediamine dihydrochloride (NEDD) and 1% sulfanilamide in 5% phosphoric acid. Nitrate reductase (Aspergillus species) and L-glutamate dehydrogenase (Candida utilus) are commercially available. Cadmium powder (100 mesh) can be purchased from Aldrich (Milwaukee, Wis.) and should be treated as outlined in Green et al. with the following modifications. A small amount of cadmium (2-3 g) should be washed with water in a 250 ml Erlenmeyer flask to remove fines. The cadmium should then be washed 2 times with 2% CuSO₄.H₂O. The washes should be done quickly, as the cadmium will turn a reddish-brown color if left in the solution too long, resulting in a lowered reduction capacity. The copper-plated cadmium should be decanted and washed copiously with water, followed by 1% phosphoric acid. The copper-plated cadmium should be stored in 1% phosphoric acid with as little headspace as possible.

In a disposable semimicrocuvette the following should be combined: 210 μl cell culture supernatant, 60 milliunits Nitrate reductase, and 25 μM NADPH. The sample should be incubated for 30 minutes at room temperature to reduce NO₃⁻ to NO₂⁻, and then the following should be added: 200 milliunits L-Glutamate dehydrogenase, 100 mM NH₄Cl, and 4 mM freshly prepared α-ketoglutarate. The sample should then be incubated at room temperature for another 10 minutes to allow for any residual NADPH to be consumed, as this interferes with the Griess reaction. Then, 250 μl of Griess reagent should be added, bringing the total reaction volume up to 500 μl. The sample should be incubated at 37° C. for 5 minutes. The absorbance at 543 nm can then be recorded versus a blank which should contain buffer and Griess reagent. Concentrations of NO₂⁻ and NO₃⁻ in the samples can be determined using a standard curve generated with known concentrations of NO₂⁻ and NO₃⁻.

### Example 3

Effect of Aminosugars on IGF-Stimulated Sulfated Glycosaminoglycan (SGAG) Synthesis

Both in vivo and in vitro basal and IGF-stimulated sulfated glycosaminoglycan (SGAG) synthesis can be determined by a method similar to that outlined elsewhere (Scharstuhl et al. Ann. Rheum. Dis. 61: 1095). The level of ³⁵S-sodium sulfate (ICN Radiochemicals, Irvine, Calif.) incorporation in SGAG can be measured in the extracellular fraction. This can be done both in the presence of and absence of the aminosugar derivatives of the present invention, including, but not limited to derivatives of glucosamine, derivatives of galactosamine, derivatives of cyclitol, and derivatives of iminocyclitol.

In Vivo SGAG Synthesis:

Right knee joints of mice aged three months or 18 months can be injected with IGF in PBS supplemented with 0.1% bovine serum albumin and an aminosugar derivative of the present invention, including, but not limited to derivatives of glucosamine, derivatives of galactosamine, derivatives of cyclitol, and derivatives of iminocyclitol. Injections can be given three times on alternate days (days 1, 3, and 5). The left knee joints can be injected with IGF in PBS supplemented with 0.1% bovine serum albumin and will serve as an internal control.

Synthesis of SGAG can be measured ex vivo, one day after the last injection. Mice should be killed and the patella with a standard amount of surrounding tissue should be dissected as described elsewhere (van den Berg et al. Rheumatol Int. 6: 273). Patellae should then be pulse labeled with ³⁵S-sulphate (20 μCi, two hours, 37° C.). This should then be followed by washing the patellae extensively with physiological saline and fixing in 96% ethanol for 24 hours at room temperature. Patellae should then be decalcified in 5% formic acid for four hours at room temperature. Then, articular cartilage should be stripped from the under-lying bone and dissolved in Lumasolve at 60° C. (Lumac, Groningen, The Netherlands). For every patella the incorporation of ³⁵S should be measured separately using a liquid scintillation counter.

In Vitro SGAG Synthesis:

Mice should be killed between the ages of three and five months or 12 and 21 months. The patella with surrounding tissue should be dissected in standardized fashion and placed in RPMI1640 medium (Dutchmodification, Flowlaboratories, Irvine, UK). Patellae should be divided into at least six treatment groups (minimum six patellae in each group): (1) no treatment, (2) IGF, (3) IGF+a derivative of glucosamine, (4) IGF+a derivative of galactosamine, (5) IGF+a derivative of cyclitol, and (6) IGF+a derivative of iminocyclitol. The tissue culture medium should be changed every 24 hours. After 48 hours of treatment, patellae should be labeled for two hours with 20 μCi radioactive sulphate in RPMI1640 (Dutchmodification) supplemented with gentamicine (50 mg/l), 2 mM/L-glutamine at 37° C., and 5% CO2. The rest of the labeling procedure should be carried out as described above. All absorbances and ³⁵S incorporation levels can be transformed to percentages compared with control treatment (=100%). Results can be statistically analyzed by analysis of variance (ANOVA).

### Example 4

Effect of Aminosuzars on IL1 Induced IL-6 and Matrix Metalloproteinases (MMPs) in Cultured Chondrocytes

Measurement of IL-6 in Cultured Chondrocytes by Protein ELISA:

Cultured cells, such as the osteoarthritic human articular chondrocytes described in Example 1 above, can be plated at 40,000 cells/well in 96-well plates in the presence of 1% FBS. After 48 hours the medium will be changed. The cells should then be stimulated with 5 ng/ml IL-1 for 24 hours in the presence of the aminosugar derivatives of the present invention, including, but not limited to derivatives of glucosamine, derivatives of galactosamine, derivatives of cyclitol, and derivatives of iminocyclitol. The IL-6 level in the culture supernatants will be measured by protein ELISA (R&D Systems, Minneapolis, Minn.) in accordance with the supplier's protocol. Results should be read at 450 mn.

Measurement of MMPs in Cultured Chondrocytes by Protein ELISA:

Interleukin 1 (IL1) is considered to be one of the most important catabolic factors in joint diseases. In OA large quantities of IL1 are produced by chondrocytes, leading to the production of cartilage degrading matrix metalloproteinases (MMPs).

Cultured cells, such as the osteoarthritic human articular chondrocytes described in Example 1 above, can be plated at 40,000 cells/well in 96-well plates in the presence of 1% FBS. After 48 hours the medium will be changed. The cells should then be stimulated with 5 ng/ml IL-1 for 24 hours in the presence of the aminosugar derivatives of the present invention, including, but not limited to derivatives of glucosamine, derivatives of galactosamine, derivatives of cyclitol, and derivatives of cyclitol. IL-1 induced MMP-1 to -13 will be measured by protein ELISA (Amersham Biosciences) and of particular interest are MMP-3 and MMP-13.

### Other Embodiments

All references discussed above are herein incorporated by reference in their entirety for all purposes. While this invention has been particularly shown and described with references to preferred embodiments thereof, it will be understood by those skilled in the art that various changes in form and details may be made therein without departing from the spirit and scope of the invention as defined by the appended claims.

We claim:

1. A method of treating an osteoarthritis related disorder in a mammal comprising administering a compound to said mammal, wherein said compound further comprises a therapeutically effective amount of an aminosugar derivative, wherein said aminosugar derivative is selected from the group consisting of a derivative of glucosamine, a derivative galactosamine, a derivative of cyclitol, a derivative of iminocyclitol, and pharmaceutically acceptable salts thereof.

2. The method according to claim 1, wherein said osteoarthritis related disorder is selected from the group consisting of osteoarthritis, rheumatoid arthritis, synovitis, subchondral bone edema, and cartilage degradation.

3. The method according to claim 2, wherein said osteoarthritis related disorder is osteoarthritis.

4. The method according to claim 2, wherein said osteoarthritis related disorder is rheumatoid arthritis.

5. The method according to claim 2, wherein said osteoarthritis related disorder is synovitis.

6. The method according to claim 2, wherein said osteoarthritis related disorder is subchondral bone edema.

7. The method according to claim 2, wherein said osteoarthritis related disorder is cartilage degradation.

8. The method according to claim 1, wherein said aminosugar derivative is a derivative of glucosamine or a pharmaceutically acceptable salt thereof.

9. The method according to claim 8, wherein said derivative of glucosamine is selected from the group consisting of compounds of formula V wherein: 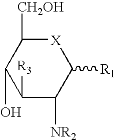
X is O;
R₁ is selected from the group consisting of: methoxy,benzyloxy,p-nitrophenoxy, hydroxyl, 5-bromo4-chloro-indolyl, tetradecanoyl-BSA, and aminitol;
R₂ is selected from the group consisting of: acetyl, benzoyl, trifluoroacetyl, aminoacetyl, and butyryl; and
R₃ is selected from the group consisting of: hydroxyl, (R)-1-carboxyethyl, and 1-carboxyethyloxy.

10. The method according to claim 8, wherein said derivative of glucosamine is a derivative of N-acetyl glucosamine or a pharmaceutically acceptable salt thereof.

11. The method according to claim 10, wherein said derivative of N-acetyl glucosamine is selected from the group consisting of compounds of formula V wherein: 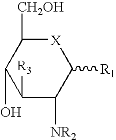
X is O;
R₁ is selected from the group consisting of: methoxy,benzyloxy, p-nitrophenoxy, hydroxyl, 5-bromo-4-chloro-indolyl, tetradecanoyl-BSA, and aminitol;
R₂ is selected from the group consisting of: acetyl, benzoyl, trifluoroacetyl, aminoacetyl, and butyryl; and
R₃ is selected from the group consisting of: hydroxyl, (R)-1-carboxyethyl, and carboxyethoxy.

12. The method according to claim 1, wherein said aminosugar derivative is a derivative of cyclitol or a pharmaceutically acceptable salt thereof.

13. The method according to claim 12, wherein said derivative of cyclitol is selected from the group consisting of compounds of formula V wherein: 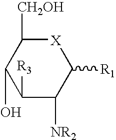
X is CH₂;
R₁ is selected from the group consisting of: methoxy,benzyloxy, p-nitrophenoxy, hydroxyl, 5-bromo4-chloro-indolyl, tetradecanoyl-BSA, and aminitol;
R₂ is selected from the group consisting of: acetyl, benzoyl, trifluoroacetyl, aminoacetyl, and butyryl; and
R₃ is selected from the group consisting of: hydroxyl, (R)-1-carboxyethyl, and 1-carboxyethyloxy.

14. The method according to claim 1, wherein said aminosugar derivative is a derivative of galactosamine or a pharmaceutically acceptable salt thereof.

15. The method according to claim 14, wherein said derivative of galactosamine is selected from the group consisting of compounds of formula VI wherein: 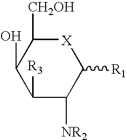
X is O;
R¹ is selected from the group consisting of: methoxy,benzyloxy, p-nitrophenoxy, hydroxyl, 5-bromo4-chloro-indolyl, tetradecanoyl-BSA, and aminitol;
R₂ is selected from the group consisting of: acetyl, benzoyl, trifluoroacetyl, aminoacetyl, and butyryl; and
R₃ is selected from the group consisting of: hydroxyl, (R)-1-carboxyethyl, and 1-carboxyethyloxy.

16. The method according to claim 1, wherein said aminosugar derivative is a derivative of iminocyclitol or a pharmaceutically acceptable salt thereof.

17. The method according to claim 12, wherein said derivative of iminocyclitol is selected from the group consisting of compounds of formula V wherein: 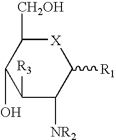
X is NH;
R¹ is selected from the group consisting of: methoxy,benzyloxy,p-nitrophenoxy, hydroxyl, 5-bromo4-chloro-indolyl, tetradecanoyl-BSA, and aminitol;
R₂ is selected from the group consisting of: acetyl, benzoyl, trifluoroacetyl, aminoacetyl, and butyryl; and
R₃ is selected from the group consisting of: hydroxyl, (R)-1-carboxyethyl, and 1-carboxyethyloxy.

18. The method according to claim 1, wherein said aminosugar derivative is selected from the group consisting of formula I, wherein: 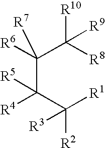
R¹ is: CHO, CH₂OH, or CO₂H;
R² is: H, OH, OR¹¹ (where R¹¹ is ether-linked cyclic or acyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group, NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R³ is: H, C-linked cyclic or acyclic alkyl, aryl, heterocyclic group, or R², R³═O;
R⁴ is: H, OH, OR¹¹ (where R¹¹ is ether-linked cyclic or acyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group, NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R⁵ is: H, C-linked cyclic or acyclic alkyl, aryl, heterocyclic group, or R⁴, R⁵═O;
R⁶ is: H, OH, OR¹¹ (where R¹¹ is ether-linked cyclic or acyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group, NH₂, NR¹⁴ R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R⁷ is: H, C-linked cyclic or acyclic alkyl, aryl, heterocyclic group, or R⁶, R⁷═O;
R⁸ is: H, OH, OR¹¹ (where R¹¹ is ether-linked cyclic or acyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group, NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R⁹ is: H, C-linked cyclic or acyclic alkyl, aryl, heterocyclic group, or R⁸, R⁹═O; and
R¹⁰ is: H, CH₃, CH₂OH, CH₂OR¹¹ (where R¹¹ is ether-linked cyclic or acyclic alkyl, aryl, heterocyclic group), CH₂OCOR¹² (where R¹² is cyclic or acyclic alkyl, aryl, heterocyclic, or amino acid derivative), CH₂Cl, CH₂Br, CH₂F, CH₂SH, CH₂SR¹³ (where R¹³ is ether-linked cyclic or acyclic alkyl, aryl, heterocyclic group, CH₂NH₂, CH₂NR¹⁴R¹⁵ (where R¹⁴ or R¹⁵ is H or ether-linked cyclic or acyclic alkyl, aryl, heterocyclic group), or CH₂NHCOR¹⁶ (where R¹⁶ is cyclic or acyclic alkyl, aryl, heterocyclic, or amino acid derivative).

19. The method according to claim 1, wherein said aminosugar derivative is selected from the group consisting of formula II, wherein: 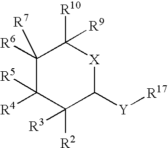
X is: O, S, CH₂, NH, or NR²⁰ (where R²⁰ is cyclic or acyclic alkyl, aryl, heteroxyclic group);
Y is: O, S, CH₂, or NH;
R¹⁷ is: H, OH, OR¹¹ (where R¹¹ is ether-linked cyclic or acyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group), NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R² is: H, OH, OR¹¹ (where R¹¹ is ether-linked cyclic or acyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group), NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R³ is: H, C-linked cyclic or acyclic alkyl, aryl, heterocyclic group, or R², R³═O;
R⁴ is: H, OH, OR¹¹ (where R¹¹ is ether-linked cyclic or acyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group), NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R⁵ is: H, C-linked cyclic or acyclic alkyl, aryl, heterocyclic group, or R⁴, R⁵═O;
R⁶ is: H, OH, OR¹¹ (where R¹¹ is ether-linked cyclic or acyclic alkyl, aryl, heterocyclic group), OCOR¹² (where R¹² is cyclic or acyclic alky, aryl, heterocyclic, or amino acid derivative), Cl, Br, F, SH, SR¹³ (where R¹³ is ether-linked cyclic or acyclic alkyl, aryl, heterocyclic group), NH₂, NR¹⁴ R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R⁷ is: H, C-linked cyclic or acyclic alkyl, aryl, heterocyclic group, or R⁶, R⁷═O;
R⁹ is: H, C-linked cyclic or acyclic alkyl, aryl, or heterocyclic group; and
R¹⁰ is: H, CH₃, CH₂OH, CH₂OR¹¹ (where R¹¹ is ether-linked cyclic or acyclic alkyl, aryl, heterocyclic group), CH₂OCOR¹² (where R¹² is cyclic or acyclic alkyl, aryl, heterocyclic, or amino acid derivative), CH₂Cl, CH₂Br, CH₂F, CH₂SH, CH₂SR¹³ (where R¹³ is ether-linked cyclic or acyclic alkyl, aryl, heterocyclic group), CH₂NH₂, CH₂NR¹⁴R¹⁵ (where R¹⁴ or R¹⁵ is H or ether-linked cyclic or acyclic alkyl, aryl, heterocyclic group), or CH₂NHCOR¹⁶ (where R¹⁶ is cyclic or acyclic alkyl, aryl, heterocyclic, or amino acid derivative).

20. The method according to claim 1, wherein said aminosugar derivative is selected from the group consisting of formula II, wherein: 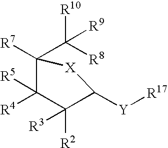
X is: O, S, CH₂, NH, or NR²⁰(where R²⁰ is cyclic or acyclic alkyl, aryl, heterocyclic group);
Y is: O, S, CH₂, or NH;
R¹⁷ is: H, OH, OR¹¹ (where R¹¹ is ether-linked cyclic or acyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group), NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R² is: H, OH, OR¹¹ (where R¹¹ is ether-linked cyclic or acyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group), NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R³ is: H, C-linked cyclic or acyclic alkyl, aryl, heterocyclic group, or R², R³═O;
R⁴ is: H, OH, OR¹¹ (where R¹¹ is ether-linked cyclic or acyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group), NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R⁵ is: H, C-linked cyclic or acyclic alkyl, aryl, heterocyclic group, or R⁴, R⁵═O;
R⁷ is: H, C-linked cyclic or acyclic alkyl, aryl, or heterocyclic group;
R⁸ is: H, OH, OR¹¹ (where R¹¹ is ether-linked cyclic or acyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group), NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R⁹ is: H, C-linked cyclic or acyclic alkyl, aryl, or heterocyclic group; and
R¹⁰ is: H, CH₃, CH₂OH, CH₂OR¹¹ (where R¹¹ is ether-linked cyclic or acyclic alkyl, aryl, heterocyclic group), CH₂OCOR¹² (where R¹² is cyclic or acyclic alkyl, aryl, heterocyclic, or amino acid derivative), CH₂Cl, CH₂Br, CH₂F, CH₂SH, CH₂SR¹³ (where R¹³ is ether-linked cyclic or acyclic alkyl, aryl, heterocyclic group), CH₂NH₂, CH₂NR¹⁴R¹⁵ (where R¹⁴ or R¹⁵ is H or ether-linked cyclic or acyclic alkyl, aryl, heterocyclic group), or CH₂NHCOR¹⁶ (where R¹⁶ is cyclic or acyclic alkyl, aryl, heterocyclic, or amino acid derivative).

21. The method according to claim 1, wherein said aminosugar derivative is selected from the group consisting of formula IV, wherein: 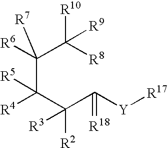
Y is: O, S, CH₂, or NH;
R¹⁷ is: H, OH, OR¹¹ (where R¹¹ is ether-linked cyclic or acyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group), NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R¹⁸ is: H, O, NH, or NR¹⁹ (where R¹⁹ is cyclic or a cyclic alkyl, aryl, heterocyclic group or acyl-linked cyclic or acyclic alkyl, aryl, or heterocyclic group);
R² is: H, OH, OR¹¹ (where R¹¹ is ether-linked cyclic or acyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group, NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R³ is: H, C-linked cyclic or acyclic alkyl, aryl, heterocyclic group, or R², R³═O;
R⁴ is: H, OH, OR¹¹ (where R¹¹ is ether-linked cyclic or acyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group, NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R⁵ is: H, C-linked cyclic or acyclic alkyl, aryl, heterocyclic group, or R⁴, R⁵═O;
R⁶ is: H, OH, OR¹¹ (where R¹¹ is ether-linked cyclic or acyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group, NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R⁷ is: H, C-linked cyclic or acyclic alkyl, aryl, heterocyclic group, or R⁶, R⁷═O;
R⁸ is: H, OH, OR¹¹ (where R¹¹ is ether-linked cyclic or acyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group, NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R⁹ is: H, C-linked cyclic or acyclic alkyl, aryl, heterocyclic group, or R⁸, R⁹═O; and
R¹⁰ is: H, CH₃, CH₂OH, CH₂OR¹¹ (where R¹¹ is ether-linked cyclic or acyclic alkyl, aryl, heterocyclic group), CH₂OCOR¹² (where R¹² is cyclic or acyclic alkyl, aryl, heterocyclic, or amino acid derivative), CH₂Cl, CH₂Br, CH₂F, CH₂SH, CH₂SR¹³ (where R¹³ is ether-linked cyclic or acyclic alkyl, aryl, heterocyclic group, CH₂NH₂, CH₂NR¹⁴R¹⁵ (where R¹⁴ or R¹⁵ is H or ether-linked cyclic or acyclic alkyl, aryl, heterocyclic group), CH₂NHCOR¹⁶ (where R¹⁶ is cyclic or acyclic alkyl, aryl, heterocyclic, or amino acid derivative).

22. The method of claim 1, wherein said aminosugar derivative further comprises anti-infammatory properties.

23. The method of claim 22, wherein said anti-inflammatory properties of said aminosugar derivative are the result of interference of said aminosugar derivative with cytokine-inducible gene expression in chondrocytes.

24. The method of claim 1, wherein said aminosugar derivative further comprises chondroprotective properties.

25. The method of claim 24, wherein said chondroprotective properties of said aminosugar derivative are the result of interference of said aminosugar derivative with cytokine-inducible gene expression in chondrocytes.

26. The method of claim 1, wherein said aminosugar derivative further comprises improved protein binding.

27. The method of claim 26, wherein said protein is an intracellular receptor.

28. The method of claim 26, wherein said protein is an extracellular recptor.

29. The method of claim 1, wherein said aminosugar derivative further comprises improved penetration of the chrondrocytes.

30. The method of claim 1, wherein said aminosugar derivative further comprises increased hydrophobicity.

31. The method of claim 1, wherein said aminosugar derivative is administered to a mammal by means selected from the group consisting of intra-articular administration, topical administration, and intra-muscular administration.

32. The method of claim 31, wherein said administration of said aminosugar derivative is by intra-articular administration.

33. The method of claim 32, wherein said administration of said aminosugar derivative is by intra-articular administration as a controlled release formula.

34. The method of claim 33, wherein said aminosugar derivative is administered by intra-articular administration while contained in a matrix as a controlled release formulation.

35. The method of claim 32, wherein said intra-articular administration of said aminosugar derivative results in retardation of cartilage degeneration.

36. The method of claim 32, wherein said intra-articular administration of said aminosugar derivative results in reduction of synovial membrane inflammation.

37. The method of claim 36, wherein said reduction of synovial membrane inflammation occurs at the macroscopic level.

38. The method of claim 36, wherein said reduction of synovial membrane inflammation occurs at the microscopic level.

39. The method of claim 31 wherein said administration of said aminosugar derivative is by topical administration.

40. The method of claim 31, wherein said administration of said aminosugar derivative is by intra-muscular administration.

41. The method of claim 1, wherein said aminosugar derivative is administered in combination with an anti-inflammatory drug.

42. The method of claim 1, wherein said aminosugar derivative is administered in combination with a hexosaminidase inhibitor.

43. The method of claim 1, wherein said method of treating said condition is selected from the group consisting of treatment of said condition, prevention of said condition, and lessening the severity of said condition.

44. The method of claim 43, wherein said method of treating said condition consists of treatment of said condition.

45. The method of claim 43, wherein said method of treating said condition consists of prevention of said condition.

46. The method of claim 43, wherein said method of treating said condition consists of lessening the severity of said condition.

47. A formulation for the treatment of osteoarthritis related disorders comprising a compound, wherein said compound further comprises a therapeutically effective amount of an aminosugar derivative, wherein said aminosugar derivative is selected from the group consisting of a derivative of glucosamine, a derivative galactosamine, a derivative of a cyclitol, a derivative of iminocyclitol, and pharmaceutically acceptable salts thereof.

48. The formulation according to claim 47, wherein said osteoarthritis related disorder is selected from the group consisting of osteoarthritis, rheumatoid arthritis, synovitis, subchondral bone edema, and cartilage degradation.

49. The formulation according to claim 48, wherein said osteoarthritis related disorder is osteoarthritis.

50. The formulation according to claim 48, wherein said osteoarthritis related disorder is rheumatoid arthritis.

51. The formulation according to claim 48, wherein said osteoarthritis related disorder is synovitis.

52. The formulation according to claim 48, wherein said osteoarthritis related disorder is subchondral bone edema.

53. The formulation according to claim 48, wherein said osteoarthritis related disorder is cartilage degradation.

54. The formulation according to claim 47, wherein said aminosugar derivative is a derivative of glucosamine or a pharmaceutically acceptable salt thereof.

55. The formulation according to claim 54, wherein said derivative of glucosamine is selected from the group consisting of compounds of formula V wherein: 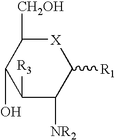
X is O;
R¹ is selected from the group consisting of: methoxy,benzyloxy, p-nitrophenoxy, hydroxyl, 5-bromo-4-chloro-indolyl, tetradecanoyl-BSA, and aminitol;
R₂ is selected from the group consisting of: acetyl, benzoyl, trifluoroacetyl, aminoacetyl, and butyryl; and
R₃ is selected from the group consisting of: hydroxyl, (R)-1-carboxyethyl, and 1-carboxyethyloxy.

56. The formulation according to claim 54, wherein said derivative of glucosamine is a derivative of N-acetyl glucosamine or a pharmaceutically acceptable salt thereof.

57. The formulation according to claim 56, wherein said derivative of N-acetyl glucosamine is selected from the group consisting of compounds of formula V wherein: 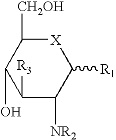
X is O;
R₁ is selected from the group consisting of: methoxy,benzyloxy, p-nitrophenoxy, hydroxyl, 5-bromo-4-chloro-indolyl, tetradecanoyl-BSA, and aminitol;
R₂ is selected from the group consisting of: acetyl, benzoyl, trifluoroacetyl, aminoacetyl, and butyryl; and
R₃ is selected from the group consisting of: hydroxyl, (R)-1-carboxyethyl, and carboxyethoxy.

58. The formulation according to claim 47, wherein said aminosugar derivative is a derivative of cyclitol or a pharmaceutically acceptable salt thereof.

59. The formulation according to claim 58, wherein said derivative of cyclitol is selected from the group consisting of compounds of formula V wherein: 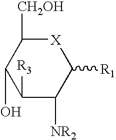
X is CH₂
R₁ is selected from the group consisting of: methoxy,benzyloxy, p-nitrophenoxy, hydroxyl, 5-bromo-4-chloro-indolyl, tetradecanoyl-BSA, and aminitol;
R₂ is selected from the group consisting of: acetyl, benzoyl, trifluoroacetyl, aminoacetyl, and butyryl; and
R₃ is selected from the group consisting of: hydroxyl, (R)-1-carboxyethyl, and 1-carboxyethyloxy.

60. The formulation according to claim 47, wherein said aminosugar derivative is a derivative of galactosamine or a pharmaceutically acceptable salt thereof.

61. The formulation according to claim 60, wherein said derivative of galactosamine is selected from the group consisting of compounds of formula VI wherein: 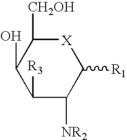
X is O;
R₁ is selected from the group consisting of: methoxy, benzyloxy, p-nitrophenoxy, hydroxyl, 5-bromo4-chloro-indolyl, tetradecanoyl-BSA, and aminitol;
R₂ is selected from the group consisting of: acetyl, benzoyl, trifluoroacetyl, aminoacetyl, and butyryl; and
R₃ is selected from the group consisting of: hydroxyl, (R)-1-carboxyethyl, and 1-carboxyethyloxy.

62. The method according to claim 1, wherein said aminosugar derivative is a derivative of iminocyclitol or a pharmaceutically acceptable salt thereof.

63. The method according to claim 12, wherein said derivative of iminocyclitol is selected from the group consisting of compounds of formula V wherein: 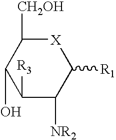
X is NH;
R₁ is selected from the group consisting of: methoxy, benzyloxy, p-nitrophenoxy, hydroxyl, 5-bromo-4-chloro-indolyl, tetradecanoyl-BSA, and aminitol;
R₂ is selected from the group consisting of: acetyl, benzoyl, trifluoroacetyl, aminoacetyl, and butyryl; and
R₃ is selected from the group consisting of: hydroxyl, (R)-1-carboxyethyl, and 1-carboxyethyloxy.

64. The formulation according to claim 47, wherein said aminosugar derivative is selected from the group consisting of formula I, wherein: 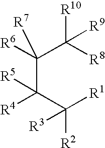
R₁ is: CHO, CH₂OH, or CO₂H;
R² is: H, OH, OR¹¹ (where R¹¹ is ether-linked cyclic or acyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group, NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R³ is: H, C-linked cyclic or acyclic alkyl, aryl, heterocyclic group, or R², R³═O;
R⁴ is: H, OH, OR¹¹ (where R¹¹ is ether-linked cyclic or acyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group, NH₂, NR¹⁴ R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R⁵ is: H, C-linked cyclic or acyclic alkyl, aryl, heterocyclic group, or R⁴, R⁵═O;
R⁶ is: H, OH, OR¹¹ (where R¹¹ is ether-linked cyclic or acyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group, NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R⁷ is: H, C-linked cyclic or acyclic alkyl, aryl, heterocyclic group, or R⁶, R⁷═O;
R⁸ is: H, OH, OR¹¹ (where R¹¹ is ether-linked cyclic or acyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group, NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R⁹ is: H, C-linked cyclic or acyclic alkyl, aryl, heterocyclic group, or R⁸, R⁹═O; and
R¹⁰ is: H, CH₃, CH₂OH, CH₂OR¹¹ (where R¹¹ is ether-linked cyclic or acyclic alkyl, aryl, heterocyclic group), CH₂OCOR¹² (where R¹² is cyclic or acyclic alkyl, aryl, heterocyclic, or amino acid derivative), CH₂Cl, CH₂Br, CH₂F, CH₂SH, CH₂SR¹³ (where R¹³ is ether-linked cyclic or acyclic alkyl, aryl, heterocyclic group, CH₂NH₂, CH₂NR¹⁴R¹⁵ (where R¹⁴ or R¹⁵ is H or ether-linked cyclic or acyclic alkyl, aryl, heterocyclic group), or CH₂NHCOR¹⁶ (where R¹⁶ is cyclic or acyclic alkyl, aryl, heterocyclic, or amino acid derivative).

65. The formulation according to claim 47, wherein said aminosugar derivative is selected from the group consisting of formula II, wherein: 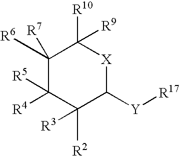
X is: O, S, CH₂, NH, or NR²⁰ (where R²⁰ is cyclic or acyclic alkyl, aryl, heteroxyclic group);
Y is: O, S, CH₂, or NH;
R¹⁷ is: H, OH, OR¹¹ (where R¹¹ is ether-linked cyclic or acyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group), NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R² is: H, OH, OR¹¹ (where R¹¹ is ether-linked cyclic or acyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group), NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R³ is: H, C-linked cyclic or acyclic alkyl, aryl, heterocyclic group, or R², R³═O;
R⁴ is: H, OH, OR¹¹ (where R¹¹ is ether-linked cyclic or acyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group), NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R⁵ is: H, C-linked cyclic or acyclic alkyl, aryl, heterocyclic group, or R⁴, R⁵═O;
R⁶ is: H, OH, OR¹¹ (where R¹¹ is ether-linked cyclic or acyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group), NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R⁷ is: H, C-linked cyclic or acyclic alkyl, aryl, heterocyclic group, or R⁶, R⁷═O;
R⁹ is: H, C-linked cyclic or acyclic alkyl, aryl, or heterocyclic group; and
R¹⁰ is: H, CH₃, CH₂OH, CH₂OR¹¹ (where R¹¹ is ether-linked cyclic or acyclic alkyl, aryl, heterocyclic group), CH₂OCOR¹² (where R¹² is cyclic or acyclic alkyl, aryl, heterocyclic, or amino acid derivative), CH₂Cl, CH₂Br, CH₂F, CH₂SH, CH₂SR¹³ (where R¹³ is ether-linked cyclic or acyclic alkyl, aryl, heterocyclic group), CH₂NH₂, CH₂NR¹⁴R¹⁵ (where R¹⁴ or R¹⁵ is H or ether-linked cyclic or acyclic alkyl, aryl, heterocyclic group), or CH₂NHCOR¹⁶ (where R¹⁶ is cyclic or acyclic alkyl, aryl, heterocyclic, or amino acid derivative).

66. The formulation according to claim 47, wherein said aminosugar derivative is selected from the group consisting of formula III, wherein: 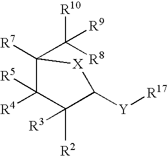
X is: O, S, CH₂, NH, or NR²⁰ (where R²⁰ is cyclic or acyclic alkyl, aryl, heterocyclic group);
Y is: O, S, CH₂, or NH;
R¹⁷ is: H, OH, OR¹¹ (where R¹¹ is ether-linked cyclic or acyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group), NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R² is: H, OH, OR¹¹ (where R¹¹ is ether-linked cyclic or acyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group), NH₂, NR¹⁴ R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R³ is: H, C-linked cyclic or acyclic alkyl, aryl, heterocyclic group, or R², R³═O;
R⁴ is: H, OH, OR¹¹ (where R¹¹ is ether-linked cyclic or acyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group), NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R⁵ is: H, C-linked cyclic or acyclic alkyl, aryl, heterocyclic group, or R⁴, R⁵═O;
R⁷ is: H, C-linked cyclic or acyclic alkyl, aryl, or heterocyclic group;
R⁸ is: H, OH, OR¹¹ (where R¹¹ is ether-linked cyclic or acyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group), NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amnino acid derivative);
R⁹ is: H, C-linked cyclic or acyclic alkyl, aryl, or heterocyclic group; and
R¹⁰ is: H, CH₃, CH₂OH, CH₂OR¹¹ (where R¹¹ is ether-linked cyclic or acyclic alkyl, aryl, heterocyclic group), CH₂OCOR¹² (where R¹² is cyclic or acyclic allyl, aryl, heterocyclic, or amino acid derivative), CH₂Cl, CH₂Br, CH₂F, CH₂SH, CH₂SR¹³ (where R¹³ is ether-linked cyclic or acyclic alkyl, aryl, heterocyclic group), CH₂NH₂, CH₂NR¹⁴R¹⁵ (where R¹⁴ or R¹⁵ is H or ether-linked cyclic or acyclic alkyl, aryl, heterocyclic group), or CH₂NHCOR¹⁶ (where R¹⁶ is cyclic or acyclic alkyl, aryl, heterocyclic, or amino acid derivative).

67. The formulation according to claim 47, wherein said aminosugar derivative is selected from the group consisting of formula IV, wherein: 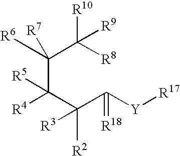
Y is: O, S, CH₂, or NH;
R¹⁷ is: H, OH, OR¹¹ (where R¹¹ is ether-linked cyclic or acyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group), NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R¹⁸ is: H, O, NH, or NR¹⁹ (where R¹⁹ is cyclic or a cyclic alkyl, aryl, heterocyclic group or acyl-linked cyclic or acyclic alkyl, aryl, or heterocyclic group);
R² is: H, OH, OR¹¹ (where R¹¹ is ether-linked cyclic or acyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group, NH₂, NR¹⁴R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R³ is: H, C-linked cyclic or acyclic alkyl, aryl, heterocyclic group, or R², R³═O;
R⁴ is: H, OH, OR¹¹ (where R¹¹ is ether-linked cyclic or acyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group, NH₂, NR¹⁴R¹⁵ (where R¹⁴ or R¹⁵ is H or ether-linked cyclic or acyclic allyl, aryl, heterocyclic group), or NHCOR¹⁶ (where R¹⁶ is cyclic or acyclic alkyl, aryl, heterocyclic, or amino acid derivative);
R⁵ is: H, C-linked cyclic or acyclic alkyl, aryl, heterocyclic group, or R⁴, R⁵═O;
R⁶ is: H, OH, OR¹¹ (where R¹¹ is ether-linked cyclic or acyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group, NH₂, NR₁₄R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R⁷ is: H, C-linked cyclic or acyclic alkyl, aryl, heterocyclic group, or R⁶, R⁷═O;
R⁸ is: H, OH, OR¹¹ (where R¹¹ is ether-linked cyclic or acyclic alkyl, aryl, heterocyclic group), OCOR¹² (where R¹² is cyclic or acyclic alkyl, aryl, heterocyclic, or amino acid derivative), Cl, Br, F, SH, SR¹³ (where R¹³ is ether-linked cyclic or acyclic alkyl, aryl, heterocyclic group, NH₂, NR14R¹⁵ (where R¹⁴ or R¹⁵ is H or ether-linked cyclic or acyclic alkyl, aryl, heterocyclic group), or NHCOR¹⁶ (where R¹⁶ is cyclic or acyclic alkyl, aryl, heterocyclic, or amino acid derivative);
R⁹ is: H, C-linked cyclic or acyclic alkyl, aryl, heterocyclic group, or R⁸, R⁹═O; and
R¹⁰ is: H, CH₃, CH₂OH, CH₂OR¹¹ (where R¹¹ is ether-linked cyclic or acyclic alkyl, aryl, heterocyclic group), CH₂OCOR¹² (where R¹² is cyclic or acyclic alkyl, aryl, heterocyclic, or amino acid derivative), CH₂Cl, CH₂Br, CH₂F, CH₂SH, CH₂SR¹³ (where R¹³ is ether-linked cyclic or acyclic alky, aryl, heterocyclic group, CH₂NH₂, CH₂NR¹⁴R¹⁵ (where R¹⁴ or R¹⁵ is H or ether-linked cyclic or acyclic alkyl, aryl, heterocyclic group), CH₂NHCOR¹⁶ (where R¹⁶ is cyclic or acyclic alkyl, aryl, heterocyclic, or amino acid derivative).

68. The formulation of claim 47, wherein said aminosugar derivative further comprises anti-infammatory properties.

69. The formulation of claim 68, wherein said anti-inflammatory properties of said aminosugar derivative are the result of interference of said aminosugar with cytokine-inducible gene expression in chondrocytes.

70. The formulation of claim 47, wherein said aminosugar derivative further comprises chondroprotective properties.

71. The formulation of claim 70, wherein said chondroprotective properties of said aminosugar derivative are the result of interference of said aminosugar with cytokine-inducible gene expression in chondrocytes.

72. The formulation of claim 47, wherein said aminosugar derivative further comprises improved protein binding.

73. The formulation of claim 72, wherein said protein is an intracellular receptor.

74. The formulation of claim 72, wherein said protein is an extracellular receptor.

75. The formulation of claim 47, wherein said aminosugar derivative further comprises improved penetration of the chrondrocytes.

76. The formulation of claim 47, wherein said aminosugar derivative further comprises increased hydrophobicity.

77. The formulation of claim 47, wherein said aminosugar derivative is administered to a mammal by means selected from the group consisting of intra-articular administration, topical administration, and intra-muscular administration.

78. The formulation of claim 77, wherein said administration of said aminosugar derivative is by intra-articular administration.

79. The formulation of claim 78, wherein said administration of said aminosugar derivative is by intra-articular administration as a controlled release formula.

80. The formulation of claim 79, wherein said aminosugar derivative is administered by intra-articular administration while contained in a matrix as a controlled release formulation.

81. The formulation of claim 78, wherein said intra-articular administration of said aminosugar derivative results in retardation of cartilage degeneration.

82. The formulation of claim 78, wherein said intra-articular administration of said aminosugar results in reduction of synovial membrane inflammation.

83. The formulation of claim 82, wherein said reduction of synovial membrane inflammation occurs at the macroscopic level.

84. The formulation of claim 82, wherein said reduction of synovial membrane inflammation occurs at the microscopic level.

85. The formulation of claim 77 wherein said administration of said aminosugar derivative is by topical administration.

86. The formulation of claim 77, wherein said administration of said aminosugar derivative is by intra-muscular administration.

87. The formulation of claim 47, wherein said aminosugar derivative is administered in combination with an anti-inflammatory drug.

88. The formulation of claim 47, wherein said aminosugar derivative is administered in combination with a hexosaminidase inhibitor.

89. The formulation of claim 47, wherein said treatment of said osteoarthritis related disorders is selected from the group consisting of treating said osteoarthritis related disorders, preventing said osteoarthritis related disorders, and lessening the severity of said osteoarthritis related disorders.

90. The formulation of claim 89, wherein said treatment consists of treating said osteoarthritis related disorders.

91. The formulation of claim 89, wherein said treatment consists of preventing said osteoarthritis related disorders.

92. The formulation of claim 89, wherein said treatment consists of lessening the severity of said osteoarthritis related disorders.
